(19) United States
(12) Patent Application Publication
(10) Pub. No.: US 2012/0059266 A1
(43) Pub. Date: Mar. 8, 2012

(54) IMAGING METHOD

(75) Inventors: Paul James Davis, England (GB); Steve Edwards, Bedfordshire (GB)

(73) Assignee: Mologic Ltd.

(21) Appl. No.: 13/255,657

(22) PCT Filed: Mar. 9, 2010
(86) PCT No.: PCT/GB2010/000421

§ 371 Date: Nov. 21, 2011

(30) Foreign Application Priority Data
Mar. 9, 2009 (GB) 0904080.9

(51) Int. Cl. A61B 6/00; A61B 10/02

(52) U.S. Cl. 600/476

(57) ABSTRACT

A docking station for use in combined imaging of a tissue wound and a test substrate comprising a sample from a tissue wound, the docking station comprises means for connecting the station to a processor which processes and stores the images. The docking station also incorporates means for receiving a test substrate comprising a sample from a tissue wound. The docking station also includes means for docking a sensor in the station, which sensor detects the light reflected from an illuminated tissue wound, such that an image of the tissue wound can be communicated from the station to the processor. The means for docking is arranged such that when the sensor is docked in the station and the test substrate is received by the docking station, the sensor is positioned to detect the intensity of reflected light from the test substrate and communicates the detected intensity of reflected light to the processor to thus permit combined imaging of the tissue wound and test substrate. An apparatus for use in combined imaging of a tissue wound and a test substrate comprising a sample from a tissue wound, comprises such a docking station together with a sensor which detects the light reflected from a tissue wound and test substrate when illuminated and a test substrate for receiving a sample from a tissue wound. A method of imaging a wound comprises directing light over a wavelength range of less than 50 nm onto the wound (9). The light reflected from the wound (9) is detected with a sensor (5) that is sensitive to the intensity of the reflected light. The intensity of the reflected light is measured.

### FIELD OF THE INVENTION

The present invention relates to wound imaging. More specifically, the invention relates to a method of imaging a wound by directing light onto the wound and corresponding devices and apparatus for performing the methods.

### BACKGROUND

Wound diagnosis is a significant aspect of the therapeutic process. The measurement of the size of a wound is important to monitor the healing progress of the wound. Wounds that do not heal at the expected rate are identified as chronic wounds and require further treatment as soon as possible. It is common for the size of a wound to be measured using a ruler, or by tracing the outline of a wound and manually or electronically calculating the area of the tracing. These techniques are cheap and easy to use, but they are reliant on an individual accurately defining the boundary of a wound, which can be difficult. Furthermore, these measurements involve direct contact with the wound, which can increase the risks of infection and be painful for the patient.

It is also important to analyse the colour of wounds or skin lesions to evaluate wound repair. It is known to take colour images of wounds in order to obtain such information non-invasively and keep a permanent record of it. A series of images can be used to detect a change in the size or colour of a wound, which is indicative of the healing or deterioration of a wound. The images can also provide further analytical information about the blood flow, redness, moisture and temperature of the wound area.

One such technique is stereophotogrammetry, which can be used to measure the area and volume of a wound. This method utilises a specialised stereo camera that can take two photographs of the wound from different perspectives and use them to create a 3D scan of the lesion in order to measure the area and volume of the wound accurately, for example the “Measurement of Area and Volume InStrument” (MAVISII).

However, these imaging techniques use digital cameras under ambient light conditions to produce colour images of the wound. The colour images are affected by changes in the colour temperature, which is dependent on the source of white light. As most white light sources get older the quality of their light changes and so causes a shift in the wavelength of the light towards the blue end of the spectrum. A change in colour temperature changes the relative amounts of red, green and blue light emitted, and so varies the relative power of the constituent wavelengths of the light.

Digital cameras will generally automatically adjust the true colour balance and brightness of an image to give an average colour intensity over the whole image. This is controlled by software parameters set in the camera. Therefore it is not possible to determine the absolute colour of an image generated by such a camera because it is distorted by this compensation, and so the imaging technique does not yield reliable data. This can be partially overcome by using carefully controlled and reproducible white light illumination. However, the problem with this approach is that, in practice, it is difficult and expensive to carry out, especially in a clinical or domestic environment.

Some expensive digital colour cameras allow this automatic adjustment of true colour balance to be switched off. However, even in such cameras the colour sensor comprises three individual red, green and blue sensors, each fitted with a specific colour filter and therefore sensitive to light of a particular wavelength so that the white light illumination still needs to be carefully controlled. In this regard, slight changes in the colour temperature of the illumination can cause significant changes in the ratio of the three red, green and blue signals that come from each sensor.

Another problem in illuminating wounds with white light is that white light illumination is, in fact, a mixture of light of wavelengths between 400 nm and 700 nm. Wounds typically are comprised of a variety of compounds which absorb light at different wavelengths. Therefore an image of a wound produced under white light illumination and detected by red, green and blue sensors does not provide clean, reliable analytical data.

A separate area of study is reported in WO 98/22023 which reports on a method for measuring skin histology. More specifically, the method involves measuring the presence and depth of dermal invasion of melanin which can give an indication as to the status of skin cancer within a patient. The method involved measuring infrared radiation from a plurality of locations over an area of skin under investigation in order to determine papillary dermis thickness and skin colour coordinates over the area. However, WO 98/22023 does not relate to the imaging of wounds (i.e. where there is a loss of the dermatological barrier function) and thus does not acknowledge the problem of producing reliable imaging data from wounds.

Another separate area of study is reported in Lau et al Sensors and Actuators B 98 (2004) 12-17. This publication reports on a non-reversible solid state ammonia sensor which comprises a poly(ethylene phthalate) strip on which a plurality of sensor dots comprising a sensor formulation are located. A sample is deposited on the sensor dots and allowed time to react to give rise to a colour change. The strip is then inserted into an imaging system comprising a black and white digital camera, surrounded by a red, a green and a blue LED. The system is configured to perform consecutive image capture by pulsing the red, green and blue illumination sources. However, Lau et al relates to an entirely ex-vivo sensor system and does not acknowledge the problem of imaging wounds objectively.

The present invention seeks to alleviate one or more of the above problems.

### SUMMARY OF THE INVENTION

According to a first aspect of the present invention, there is provided a method of imaging a subject indicative of a tissue wound, the tissue wound being characterised by the tissue losing its dermatological barrier function, the method comprising the steps of:
directing light over a wavelength range of less than 50 nm onto the each subject;
detecting the light reflected from the each subject with a sensor that is sensitive to the intensity of the reflected light; and
measuring the intensity of the reflected light.

Conveniently, the sensor is a digital monochrome camera. In certain embodiments, the sensor is a colour digital camera which has the automatic white light compensation feature disabled.

Preferably, the wavelength of the light is between 400 nm and 1000 nm, preferably between 750 nm and 1000 nm.

Advantageously, the method comprises directing light at a plurality of different wavelength ranges onto at least the first subject over a series of time periods and measuring the intensity of the reflected light at each time period.

Conveniently, the light directed onto at least the first subject is within a wavelength range of less than 10 nm.

Preferably, the step of measuring the intensity of the reflected light comprises generating an image of at least the first subject.

Advantageously, the light directed onto each subject is from an LED.

Conveniently, the method comprises using an integrated device comprising a light source for emitting light and the sensor.

Preferably, the device further comprises a processor for receiving a signal from the sensor encoding the intensity of light detected.

In a preferred embodiment, an image is generated of at least the first subject by the processor from the signal derived from the sensor encoding the intensity of light detected. Most preferably, the image is generated through communication between the sensor and a docking station as defined herein. In such embodiments, light is directed onto the subject and the intensity of reflected light is detected by the sensor. Subsequently, the sensor is docked in the docking station (optionally via a cradle in the docking station). The docking station facilitates transmission of the signal from the sensor to the processor, where the image is generated and/or processed. Accordingly, in these embodiments, the primary function of the docking station is facilitating the transmission of the signal from the sensor to the processor. Optionally, the docking station and processor may be integrated as one unit.

Advantageously, the device further comprises a display for displaying a result representative of the intensity of light detected.

Conveniently, the device further comprises electronic memory in communication with the sensor for storing data concerning the intensity of light detected.

Preferably, the each subject defines a plane and the method comprises directing the light onto each subject at an angle to the plane of less than 90°, preferably less than 70°.

Advantageously, the first subject is the tissue wound.

Alternatively, the first subject is a test substrate comprising a sample from the tissue wound.

Conveniently, the method further comprises imaging a second subject indicative of the tissue wound, wherein the first subject is the tissue wound and the second subject is a test substrate comprising a sample from the tissue wound.

Optionally, the method comprises imaging a test substrate comprising a sample from a tissue wound, wherein the test substrate is received within a structure of the docking station. In this embodiment, the docking station facilitates positioning of the test substrate relative to the sensor—when the sensor is docked in the station and the test substrate is received by the docking station, the sensor is positioned to detect the intensity of reflected light from the test substrate and communicates the detected intensity of reflected light to the processor. This effectively permits combined imaging of the tissue wound and test substrate as both signals can be communicated, via the docking station, from the sensor to the processor.

Preferably, the means for receiving the test substrate structure of the docking station comprises a port or slot of defined dimensions that are suitable for receiving the test substrate. Preferably, the dimensions of the structure allow secure positioning of the test substrate in the docking station. Suitable dimensions may be identified by the person skilled in the art, depending upon the test substrate in question, which may for example be an immunoassay test strip. In some embodiments, the structure may comprise a slot for receiving the test substrate. In addition, the docking station may comprise a suitable means for docking the sensor, such as a cradle for receiving the sensor. Preferably, placement of the test substrate into the structure of the docking station facilitates alignment of the sensor with the test substrate or part thereof that provides information on the sample within or on the test substrate. For example, if the test substrate comprises two test lines, placement of the test substrate into the structure of the docking station facilitates alignment of the sensor with the two test lines, enabling the sensor to detect the intensity of reflected light each test line.

Preferably, placement of the test substrate into the structure of the docking station enables the sensor to image the test substrate and generate a signal encoding the intensity of light detected. Most preferably, the docking station facilitates the transmission of the signal derived from the sensor to the processor for subsequent processing, analysis and/or storage of the data.

Preferably, the method further comprises the step of combining an indication derived from the imaging of the second subject with an image of the first subject.

Advantageously, the test substrate comprises two test lines and the method further comprises the step of determining the intensity of the reflected light from each test line.

Conveniently, the method further comprises the step of encoding the relative intensity of the reflected light from each test line as a digital result.

Preferably, the test substrate comprises an immunoassay test strip.

According to a second aspect of the present invention, there is provided a method of imaging a lateral flow immunoassay test strip comprising the steps of:
directing light over a wavelength range of less than 50 nm onto the test strip;
detecting the light reflected from the test strip with a sensor that is sensitive to the intensity of the reflected light; and
measuring the intensity of the reflected light.

It is preferred that the second aspect of the invention has one or more of the optional features of the other aspects of the invention.

According to a third aspect of the present invention there is provided a docking station for use in combined imaging of a tissue wound and a test substrate comprising a sample from a tissue wound (the tissue wound being characterised by the tissue losing its dermatological barrier function) the docking station comprising:

a) means for connecting the station to a processor which processes and stores the images
b) means for receiving a test substrate comprising a sample from a tissue wound;
c) means for docking a sensor in the station, which sensor detects the light reflected from an illuminated tissue wound, such that an image of the tissue wound can be communicated from the station to the processor (to permit communication of the image of the tissue wound), characterised in that the means for docking is arranged such that when the sensor is docked in the station and the test substrate is received by the docking station, the sensor is positioned to detect the intensity of reflected light from the test substrate and communicates the detected intensity of reflected light to the processor to thus permit combined imaging of the tissue wound and test substrate.

The means for receiving a test substrate may comprise any suitable structure which permits an image of the test substrate to be obtained by the sensor when docked into the docking system. As discussed herein, the means for receiving the test substrate may comprise, consist essentially of or consist of a port or slot dimensioned to securely position the test substrate within the (main body of the) docking station. The port or slot may incorporate suitable guides to assist with placement of the test substrate in the docking station.

The means for docking the sensor in the station is arranged such that when the sensor is docked in the station and the test substrate is received by the docking station, the sensor is positioned to detect the intensity of reflected light from the test substrate and communicates the detected intensity of reflected light to the processor (via the docking station which is connected to the processor) to thus permit combined imaging of the tissue wound and test substrate. Any suitable docking structure that securely positions the sensor relative to the test substrate may be employed. In certain embodiments, the means for docking comprises a cradle in which the sensor sits. The docking means or cradle may be adjustable in certain embodiments to accommodate different sensors.

The means for docking permits the images recorded by the sensor to be transmitted via the docking station to the processor. Thus the docking station may incorporate some means of connection allowing data from the sensor to be communicated to the processor. The docking station may also include means for charging the sensor whilst docked in the docking station. The means of connection and/or the means for charging may be integrated into the means for docking the sensor in some embodiments.

In some embodiments, the docking station further comprises one or more light sources to illuminate the test substrate. The light sources may, in some embodiments, also permit illumination of a tissue wound. As discussed herein, wavelength of the light emitted by the light source may be between 400 nm and 1000 nm, preferably between 750 nm and 1000 nm. The light source may permit light at a plurality of different wavelength ranges to be emitted, optionally over a series of time periods. The light source preferably emits light within a wavelength range of less than 10 nm. In specific embodiments, the one or more light sources comprise one or more LEDs. More specifically, the docking station may include first and second LED banks.

The docking station may incorporate a processor which process and stores the images of the tissue wound and test substrate. The processor is typically a computer such as a PC or Macintosh computer or a dedicated integrated circuit. Processing may involve integration of the two separate images to form an overall integrated diagnosis read out. The processor may also produce guidance for treatment which is determined by the integrated diagnosis read out resulting from the two images. This may involve application of a suitable algorithm to link the intensity of reflected light data for the two images to selection of an appropriate proposed treatment. This arms staff at the point of care with the relevant diagnostic information to be able to treat the wound effectively.

The processor may be connected to a display means for displaying the images. The display means may be a monitor, such as an LCD monitor.

In a related fourth aspect, the invention also provides an apparatus for use in combined imaging of a tissue wound and a test substrate comprising a sample from a tissue wound, the apparatus comprising:

a) a docking station of the invention as defined herein
b) a sensor which detects the light reflected from a tissue wound and test substrate when illuminated (as discussed herein)
c) a test substrate for receiving a sample from a tissue wound (as discussed herein).

The components of the apparatus are as defined herein. For example, in some embodiments, the sensor is a digital monochrome camera and the test substrate is an immunoassay test strip.

In some embodiments, the apparatus additionally comprises a processor which process and stores the images of the tissue wound and test substrate. The processor is typically a computer such as a PC or Macintosh computer or a dedicated integrated circuit.

The processor may be connected to a display means for displaying the images. The display means may be a monitor, such as an LCD monitor. As discussed herein the processor and/or display means may form an integral part of the docking station in some embodiments.

It is preferred that the third and fourth aspects of the present invention incorporate one or more of the optional features of the other aspects of the invention. The discussion herein therefore applies mutatis mutandis to these aspects.

According to a fifth aspect of the present invention, there is provided a device for imaging a wound comprising:
A light source to emit instant light over a wavelength range of less then 50 nm;
A sensor of detecting the intensity of the instant light when reflected; and
A processor for receiving a signal from the sensor, in response to detecting the intensity of light detected.

Preferably the device further comprises a structure for receiving a test substrate.

Conveniently, in all aspects of the invention, the test substrate comprises an assay strip comprising one, two or more than two test lines.

It is preferred that the fifth aspect of the present invention has one or more of the optional features of the other aspects of the invention.

A “wound” in this specification is an area of the skin or other tissue in which the dermatological barrier function is lost.

In this specification the “colour temperature” of a light source is determined by comparing its chromaticity (quality of colour) with a theoretical, heated black-body radiator. In general, low colour temperatures are red, and conversely, high colour temperatures are blue.

“Intensity” in this specification refers to the amount of energy in a light source.

In this specification, a “subject indicative of a tissue wound” is any subject whose image may be recorded where the image of the subject contains information about the status of the wound. Such a subject includes the wound itself and assay devices containing visible test results of samples from the wound.

### BRIEF DESCRIPTION OF DRAWINGS

In order that the present invention may be fully understood and so that further features thereof may be appreciated, embodiments of the invention will now be described, by way of example, with reference to the accompanying figures in which:

FIG. 1 shows a schematic view of a device for imaging a wound;

FIG. 2 shows a plan view of a lateral flow immunoassay; and

FIG. 3 is a perspective view of a combination of components for use in another embodiment of the invention.

### DETAILED DESCRIPTION

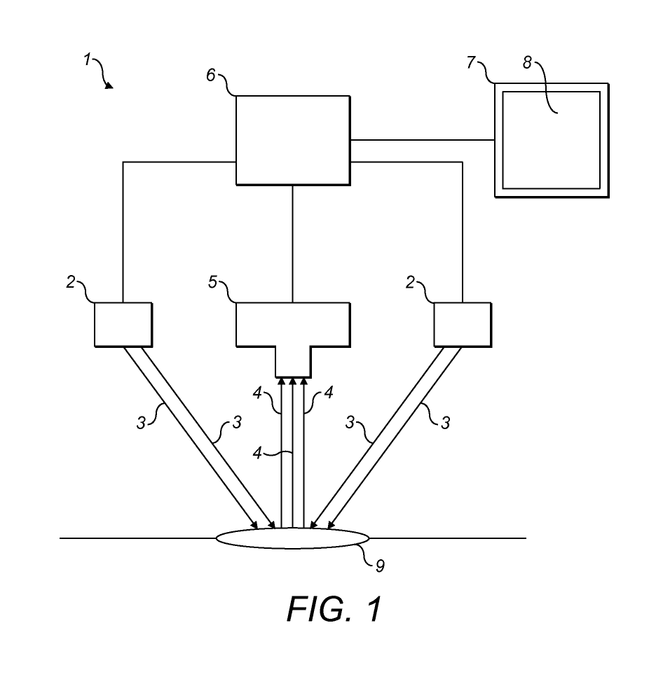

In a first embodiment of the present invention, as shown in FIG. 1, there is provided a device 1 for imaging a wound 9. The device 1 comprises first and second banks of light emitting diodes (LEDs) 2 each with a narrow band emission of 600 nm±5 nm. Located between the first and second banks of LEDs 2 there is provided a monochrome digital camera 5 which is capable of detecting light having a wavelength of between 400 nm and 1000 nm and generating a signal indicative of the intensity of the detected light. As is typical for digital cameras known in the art, the camera comprises an array of pixels, each pixel being independently sensitive to incident light and a lens arrangement for focussing incoming light on the array. The signal generated by the camera contains an indication of the intensity of the light at each pixel in the array and therefore the information necessary to generate a monochrome image of the view at which it is directed.

The first and second LED banks 2 are configured so that the light emitted therefrom is angled inwardly. The first and second LED banks 2 and the camera 5 are arranged on a plane and the light beams 3 emitted from the first and second LED banks 2 is angled inwardly at an angle of 45° to the plane such that the light beams 3 from the first and second LED banks 2 meet at a point directly in front of the camera 5.

The first and second LED banks are each separately in communication with a processor 6 such as a desktop PC or a dedicated integrated circuit. The processor is capable of controlling the intensity and timing of light emitted by the two banks of LEDs 2. The processor 6 is also capable of receiving and analysing the signal from the camera 5 and synchronising the control of the emission of light from the first and second LED banks 2 with the signal received from the camera 5. The processor 6 is in communication with electronic memory (both volatile memory such as on an integrated circuit and permanent memory such as a hard disk) (not shown) on which data from the signal from the camera 5 may be recorded, in conjunction with the status of the first and second LED banks 2 at the time that the signal from the camera 5 was received.

The processor 6 is also in communication with an LCD monitor 7 comprising a screen 8 on which an image from the camera 5 may be displayed or on which an analysis of any image from the camera 5 may be presented.

In use, the device 1 is located above a wound 9 of a patient, such as a skin wound. The device is located such that the camera 5 is directly above the wound 9 and at a distance from the wound such that the wound is at the point where the light from the first and second banks of LEDs 2 meet. The processor 6 is operated so as to instruct the first and second LED banks 2 to illuminate the wound 9. The light from the first and second LED banks 2 illuminates the wound 9. It is to be appreciated that, because the light from the first and second LED banks is angled inwardly, it illuminates the wound at an angle of approximately 45° to the plane defined by the wound 9. For this reason, light is reflected towards the camera 5 as is shown by the arrows 4 but glare from the incoming light beams 3 is minimised. The camera 5 receives the incoming light 4 and focuses it on the array of pixels as known in the art. The camera 5 generates a signal indicating the intensity of light at each of the pixels. The signal is then transmitted to the processor 6 where it is stored in the memory and processed to deliver a suitable image 8 for display on the LCD screen 7.

It is to be appreciated that because the incident light 3 on the wound 9 is of a single wavelength (or, at least, over a very narrow range of wavelengths) and the camera 5 is sensitive to light of any visible wavelength, the signal transmitted by the camera 5 is an objective and absolute record of the wound 9. Thus, in some embodiments, the images of multiple wounds taken by the same device are compared in order to provide an objective analysis and comparison of the wounds. The device 1 is particularly useful for generating images of wounds that have lost both the epidermis and the dermis layers of the skin.

It is to be appreciated that it is not essential to the invention that the first and second LED banks 2 emit light over a range of wavelengths as narrow as 10 nm. In some alternative embodiments, for example, the range of wavelengths may be up to 50 nm. Similarly, the wavelength at which the first and second LED banks emit light need not be 600 nm but can be any wavelength within the range of 400 nm to 1000 nm. Indeed, in some alternative embodiments, the LED banks 2 are capable of emitting light at more than one wavelength. For example, in one embodiment, each LED bank comprises one red, one green and one blue LED which can be illuminated independently. In these embodiments the wound 9 is illuminated by the red, green and blue LEDs, one at a time, over a series of time periods and the camera 5 records an image over each time period and transmits each image to the processor 6 where each image is stored, together with the information as to which LED was illuminating the wound 9 at the time period when the image was produced. Images of the wound 9 under red, blue and green light are then combined in the processor 6 to display an image 8 on the LCD screen 7 which represents a true image of the colours of the wound and may be compared with other “true colour” images of wounds similarly produced. Such a true absolute colour image 9 is not dependent on the colour temperature of illumination, background colour or illumination intensity.

It is to be noted that single wavelength light sources 2, such as LEDs, have stable intensity over time and hence the wavelength does not shift as the light source gets older.

In alternative embodiments, the first and second LED banks 2 are located closer to or further from the camera 5 and the angle at which the first and second LED banks 2 shine their emitted light is correspondingly adjusted so that the emitted light beams 3 still meet at a point in front of the camera 5. Nonetheless, it is generally preferred that the angle at which the wound 9 is illuminated is less than 90° to the plane defined by the wound 9 and it is particularly preferred that it is less than 70°.

It is to be understood that it is not essential to the invention that an image of the wound 9 is producible as such. In some embodiments, the signal produced by the camera 5 is processed in the processor 6 using algorithms, the results of which are displayed on the screen 7, without the displaying of any image of the wound as such.

In an alternative embodiment, the camera 5 is a colour digital camera which has the automatic white light compensation feature disabled.

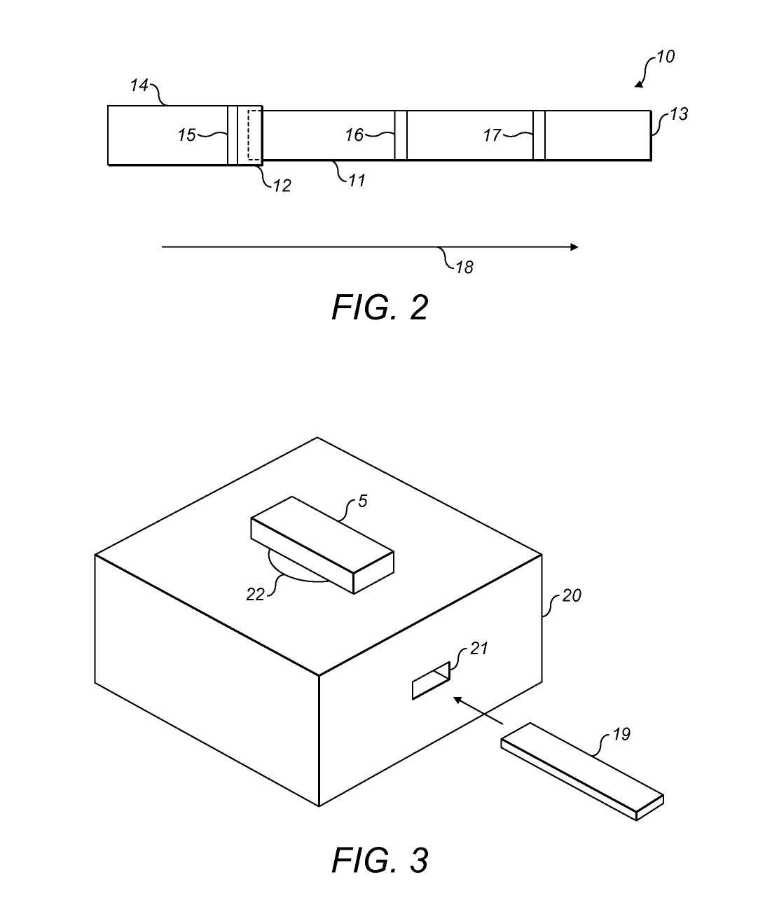

A device of the present invention may be used to generate images of subjects other than wounds. For example, in one alternative embodiment, the device 1 is used to generate an image of a test substrate. Such test substrates include lateral flow immunoassay test strip such as a pregnancy test strip. Referring to FIG. 2 a lateral flow immunoassay test strip 10 for use as a pregnancy test is shown by way of example. The test strip comprises a nitrocellulose strip 11 having upstream 12 and downstream 13 ends. Attached to the upstream end 12 is provided an absorbent sample receiving pad 14 which comprises a marker zone 15 near to where the pad 14 meets the upstream end 12. The marker zone 15 comprises a plurality of monoclonal marker antibodies, which are specific for the protein hCG and which are dried in a transverse line across the sample receiving pad 14. Each marker antibody is covalently bound to a gold particle. On the nitrocellulose strip 11, between the sample receiving pad 14 and the downstream end 13 there is provided an immobilisation zone 16. The immobilisation zone which stretches in a line across the nitrocellulose strip 11, comprises a plurality of monoclonal antibodies immobilised onto the surface of the nitrocellulose strip 11. Each of the monoclonal antibodies is capable of binding hCG at a different epitope from the epitope of the monoclonal antibodies in the marker zone 15.

Further along the nitrocellulose strip 11 in the direction of the downstream end 13 is provided a control zone 17 at which are located a plurality of monoclonal antibodies immobilised on the surface of the nitrocellulose strip 11 in a transverse line. Each of the monoclonal antibodies in the control zone 17 is capable of binding immunoglobulins.

In use, a sample (e.g. urine) is deposited on the sample receiving zone 14, where it is adsorbed along the sample receiving pad 14 towards the downstream end 13 in the direction of the arrow 18. As it passes through the marker zone 15, any hCG in the sample binds the marker antibodies which are also adsorbed along the nitrocellulose. The hCG in the sample is immobilised at the immobilisation zone 16 by the monoclonal antibodies located there. Furthermore, the marker antibodies are also immobilised at the immobilisation zone 16, with the hCG molecules acting as a bridge or linker between the antibodies. Any of the marker antibodies which are not immobilised at the immobilisation zone continue in the direction of the arrow 18 and are then immobilised at the control zone 17 by the antibodies located there. Therefore, if hCG is present in the sample then the marker antibodies are generally immobilised at the immobilisation zone 16, with only a few passing through to the control zone 17 whereas in the absence of hCG from the sample, the marker antibodies pass through the immobilisation zone 16 and are immobilised at the control zone 17. Where the marker antibodies are concentrated, the gold particle on each antibody forms a visible line.

The device of the present invention is used to generate an image of the nitrocellulose strip 11 and, more specifically, the immobilisation zone 16 and the control zone 17. In particular, the device is used to compare the intensity of the gold particle lines that form at the immobilisation and control zones 16, 17, enabling a true comparison of the zones to be carried out.

It is to be appreciated that the device of the present invention is not limited to the imaging of this particular type of immunoassay. The imaging of other test substrates is within the scope of the invention. For example, in one alternative embodiment, the method of the present invention involves generating an image of the zones of the immunoassay described in WO2007/096637, which is incorporated herein by reference.

In a further embodiment, the method involves generating an image of a wound diagnostic test strip such as is described in WO2007/128980, which is incorporated herein by reference. Also included within the term “test substrate” are test devices that do not involve lateral flow such as the enzyme detection product described in WO2007/096642, which is also incorporated herein by reference

Thus, in one embodiment, a wound 9 is illuminated by the first and second LED banks 2, as described above, and the camera 5 receives light reflected from the wound 9 and generates a signal indicating the intensity of the reflected light. The signal is transmitted to the processor 6 where it is stored in the memory. Subsequently, a sample from the wound is taken with a swab. The swab is washed in a buffer solution and the buffer solution is deposited on a test substrate 19. In this particular embodiment, the test substrate 19 is lateral flow immunoassay test strip as described in WO2007/096637. In brief, the test strip is the same as the lateral flow immunoassay test strip 10 depicted in FIG. 2 herein, except that the absorbent sample receiving pad 14 is pre-impregnated with hCG.

When the wound sample is deposited on the absorbent sample receiving pad 14, and if the sample comprises a protease enzyme which is capable of hydrolysing hCG then the hCG is broken down into one or more degradation products. Subsequently, the degradation products are adsorbed along the test strip 10 in the direction of the arrow 18 and mix with the marker antibodies in the marker zone 15. The degradation products continue along the test strip 10 but at the immobilisation zone 16, the marker antibodies are not immobilised because the degradation products of the hCG molecules are not capable of acting as a bridge or linker between the marker antibodies and the antibodies immobilised at the immobilisation zone 16. Thus the marker antibodies continue in the direction of the arrow 18, along the test strip 10, until they reach the control zone 17 at which point the marker antibodies are immobilised and a visible line forms at the control zone 17.

If, on the other hand, the wound sample does not contain a protease capable of hydrolysing the hCG molecule then the marker antibodies are immobilised at the immobilisation zone 16 with the hCG molecules acting as a bridge or linker between the marker antibodies and the monoclonal antibodies provided at the immobilisation zone 16. In this case, a visible line forms at the immobilisation zone 16. In practice, the marker antibodies are provided in excess so that even in these circumstances some marker antibodies pass through the immobilisation zone 16 and reach the control zone 17 so a visible line also forms at the control zone 17 and confirms that the assay has reached its end point.

It will be understood, therefore, that if the wound sample contains a protease capable of hydrolysing the hCG molecule a visible line forms only at the control zone 17 but if the wound sample does not contain such a protease then a visible line forms at the immobilisation zone 16 (and also at the control zone 17).

After the test strip assay 19 has reached its end point, the test substrate 19 is inserted into a docking station 20. The docking station 20 comprises a slot 21 for receiving the test substrate 19 and a cradle 22 for receiving the camera 5. Also provided in the docking station 20 are first and second LED banks (not shown).

After insertion of the test substrate 19 into the slot 21, the test substrate 19 is illuminated by the first and second LED banks. The reflected light from the test substrate 19 and, in particular, the reflected light from the immobilisation zone 16 and the control zone 17 is received by the camera 5. The image is stored in the local memory of the camera 5 before being transferred to the processor 6 where it is analysed to determine the intensity of the reflected light from each of the immobilization zone 16 and control zone 17 and, furthermore, the relative intensity of the reflected light from each zone. The relative intensity of the two zones is then encoded as a single digital result which is indicative either of the presence or absence of the protease in the wound sample or of the relative concentration of the protease in the wound sample. This digital result is then combined with the image of the wound 9 which is stored in the memory of the processor 6 to produce a consolidated data file. Thus the consolidated file comprises not only the image of the wound 9 but also the digital result indicative of the presence of, or the relative amount of, the protease in the wound sample.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

1. A docking station for use in combined imaging of a tissue wound and a test substrate comprising a sample from a tissue wound, the docking station comprising:
a) means for connecting the station to a processor which processes and stores the images
b) means for receiving a test substrate comprising a sample from a tissue wound;
c) means for docking a sensor in the station, which sensor detects the light reflected from an illuminated tissue wound, such that an image of the tissue wound can be communicated from the station to the processor, characterised in that the means for docking is arrange such that when the sensor is docked in the station and the test substrate is received by the docking station, the sensor is positioned to detect the intensity of reflected light from the test substrate and communicates the detected intensity of reflected light to the processor to thus permit combined imaging of the tissue wound and test substrate.

2. The docking station of claim 1, wherein the means for receiving a test substrate comprises a slot dimensioned to securely position the test substrate within the docking station.

3. The docking station according to claim 1, wherein the means for docking the sensor in the station comprises a cradle in which the sensor sits.

4. The docking station according to claim 1 further comprising one or more light sources to illuminate the test substrate.

5. The docking station of claim 4 wherein the one or more light sources comprise one or more LEDs.

6. The docking station of claim 5 comprising first and second LED banks.

7. An apparatus for use in combined imaging of a tissue wound and a test substrate comprising a sample from a tissue wound, the apparatus comprising:
a) a docking station according to claim 1
b) a sensor which detects the light reflected from a tissue wound and test substrate when illuminated
c) a test substrate for receiving a sample from a tissue wound.

8. The apparatus of claim 7 further comprising a processor which process and stores the images of the tissue wound and test substrate.

9. The apparatus of claim 8 wherein the sensor is a digital monochrome camera.

10. A method of imaging a tissue wound and a test substrate comprising a sample from the tissue wound, the tissue wound being characterised by the tissue losing its dermatological barrier function, the method comprising the steps of:
(a) directing light over a wavelength range of less than 50 nm onto each of the tissue wound and the test substrate comprising a sample from the tissue wound;
(b) detecting the light reflected from each of the tissue wound and the test substrate comprising a sample from the tissue wound with a sensor that is sensitive to the intensity of the reflected light; and
(c) measuring the intensity of the reflected light from each of the tissue wound and the test substrate comprising a sample from the tissue wound.

11. The method according to claim 10, wherein the sensor is a digital monochrome camera.

12. The method according to claim 10, wherein the wavelength of the light is between 400 nm and 1000 nm, preferably between 750 nm and 1000 nm.

13. The method according to claim 10 comprising directing light at a plurality of different wavelength ranges onto at least the tissue wound over a series of time periods measuring the intensity of the reflected light at each time period.

14. The method according to claim 10, wherein the light directed onto at least the tissue wound is within a wavelength range of less than 10 nm.

15. The method according to claim 10 wherein the step of measuring the intensity of the reflected light comprises generating an image of at least the tissue wound.

16. The method according to claim 10, wherein the light directed onto each of the tissue wound and the test substrate comprising a sample from the tissue wound is from an LED.

17. The method according to claim 10 wherein the method comprises using an integrated device comprising a light source for emitting light and the sensor.

18. The method according to claim 17 wherein the device further comprises
a processor for receiving a signal from the sensor encoding the intensity of light detected.

19. The method according to claim 17 wherein the device further comprises a display for displaying a result representative of the intensity of light detected.

20. The method according to claim 17 wherein the device further comprises electronic memory in communication with the sensor for storing data concerning the intensity of light detected.

21. The method according to claim 10 wherein each of the tissue wound and the test substrate comprising a sample from the tissue wound defines a plane and the method comprises directing the light onto each of the tissue wound and the test substrate comprising a sample from the tissue wound at an angle to the plane of less than 90°, preferably less than 70°.

22. The method according to claim 10 which comprises docking the sensor into a docking station as in claim 1 in order to permit detection of the intensity of reflected light from the test substrate and communication of the detected intensity of reflected light from each of the tissue wound and the test substrate comprising a sample from the tissue wound to the processor, to thus permit combined imaging of the tissue wound and test substrate.

23. The method according to claim 10 which is performed on an apparatus according to claim 7.

24. The method according to claim 10 further comprising the step of combining an indication derived from the imaging of the tissue wound with an indication derived from the imaging of the test substrate comprising a sample from the tissue wound.

25. The method according to claim 10 wherein the test substrate comprises two test lines and the method comprises the step of determining the intensity of the reflected light from each test line.

26. The method according to claim 25, further comprising the step of encoding the relative intensity of the reflected light from each test line as a digital result.

27. The method according to claim 10, wherein the test substrate comprises an immunoassay test strip.
